(19) United States
(12) Patent Application Publication
(10) Pub. No.: US 2009/0068264 A1
(43) Pub. Date: Mar. 12, 2009

(54) Clinical Applications of Tetrahydrobiopterin, Lipoic Acid and Their Salts and Methods of Preparing Tetrahydrobiopterin Bis-Lipoate

(75) Inventors: Kenneth T. Richardson, Anchorage, AK (US); Don C. Pearson, Lakewood, WA (US); John A. Edwards, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChronoRX LLC, Anchorage, AK (US)

(21) Appl. No.: 12/268,244

(22) Filed: Nov. 10, 2008

Related U.S. Application Data
(62) Division of application No. 11/326,796, filed on Jan. 6, 2006.
(60) Provisional application No. 60/643,857, filed on Jan. 14, 2005.

(51) Int. Cl. A61K 9/24; A61K 31/4985; A61P 3/10; A61P 3/00; A61P 3/06

(52) U.S. Cl. 424/472; 514/249

(57) ABSTRACT

Dosage forms and methods of use are disclosed for: a) the simultaneous administration of tetrahydrobiopterin (BH4) or a derivative, homolog or precursor thereof and lipoic acid (LA), or dihydrolipoic acid (DHLA), or a derivative, homolog or salt thereof or, b) the administration of a conjugate consisting of tetrahydrobiopterin bis-lipoate (TBL). The invention is useful for the amelioration of diabetes mellitus types 1 and 2 (including impaired glucose tolerance, pre-diabetes, insulin resistance, metabolic syndrome X and as an adjunct to oral antidiabetic agents and/or insulin), diabetic and non-diabetic microvascular diseases (including nephropathy, neuropathy and retinopathy), diabetic and non-diabetic macrovascular diseases (including heart attack, stroke, peripheral vascular disease and ischemia-reperfusion injury), hypertension, vasoconstriction, obesity, dyslipedemia, and neurodegenerative disorders (including Parkinson's disease, mild cognitive impairment, senile dementia, Alzheimer's disease, hearing loss and chronic glaucomas). A novel method for the preparation of TBL is also disclosed.

### CROSS-REFERENCE TO RELATED APPLICATIONS

This application is a division of U.S. patent application Ser. No. 11/326,796, filed Jan. 6, 2006, which claims benefit from U.S. Provisional Patent Application No. 60/643,857, filed Jan. 14, 2005. The contents of both such applications are incorporated herein by reference in their entirety. Any claims that were originally filed in application Ser. No. 11/326,796 and that have been omitted from the present application are so omitted without prejudice to being re-instituted in the present application or in a related application.

### BACKGROUND AND SUMMARY OF THE INVENTION

1. Field of the Invention

The invention is in the fields of pharmacology and biochemistry. It relates to dosage forms and methods of use for: a) the simultaneous administration of tetrahydrobiopterin (BH4), or a derivative or precursor thereof, and lipoic acid (LA), or dihydrolipoic acid or a derivative or salt thereof, or b) the administration of a conjugated molecule consisting of tetrahydrobiopterin bis-lipoate (TBL). The invention also relates to methods of preparation of TBL.

2. Description of the Prior Art and the Present Invention

For clarity within this document, the shorthand expression LABH4 indicates that lipoic acid (LA) and tetrahydrobiopterin (BH4) may be used in either the individual ingredient dosage form (‘a’ above) or in the single conjugated molecular dosage form (‘b’ above). Dosage forms and methods of use for LABH4 are described for clinical presentations of the following Clinical Targets (CTs):
1. diabetes mellitus types 1 and 2, impaired glucose tolerance, pre-diabetes, insulin resistance, metabolic syndrome X and as an adjunct to oral antidiabetic agents and/or insulin (Group A);
2. diabetic and non-diabetic microvascular diseases (Group B);
3. diabetic and non-diabetic macrovascular diseases and ischemia-reperfusion injury (Group C);
4. hypertension, vasoconstriction, including nocturnal (early AM) vasoconstriction (Group D);
5. obesity, dyslipedemia (Group E);
6. neurodegenerative disorders including Parkinson's disease, mild cognitive impairment, senile dementia, Alzheimer's disease, hearing loss and chronic glaucomas (Group F).

Although the Groups in the above list of CTs may seem wildly disparate in etiology and clinical presentation, in fact, a finite list of similar physiological and biochemical defects can be defined that are common to and link all of the Groups. The patent is designed to focus the action of its limited active ingredients upon this shared list of defects common to the CTs. This focus of the patent provides and supports the rational clinical usefulness of the invention for what may otherwise appear to be an over-broad collection of diseases. The scientific support for this design is developed below in this document.

A biological system consists of a definable set of metabolic nodes and a web of interactions between these nodes—i.e., it is a metabolic network that embraces multiple subsystems. The invention defines those metabolic subsystems, which when disturbed individually or collectively result in the pathologies targeted by the invention. The metabolic subsystems addressed by the invention are:
1) mitochondrial bioenergetics,
2) free radical modulation,
3) balance between constitutive nitric oxide & endothelin-1 levels, and
4) cellular control of calcium wave signaling. (see below, pl5, for overview).

Many, perhaps most, physiologically important and pharmacologically active substances act in a major way primarily within one metabolic subsystem. In contrast, most chronic disorders like those addressed by the invention have disturbances in all or several of their metabolic subsystems. These disturbances are often interrelated.

Remarkably, LA and BH4 are active in each of these metabolic subsystems and compliment each other, sometimes synergistically.

These relationships are reviewed again later in the document.

### I. BIOCHEMISTRY

A. Tetrahydrobiopterin (BH4)

A principal biological role for BH4 is as a cofactor for three aromatic amino acid hydroxylases: phenylalanine hydroxylase (PAH), tyrosine hydroxylase (TH), and tryptophan hydroxylase (TPH). Every aspect of these enzymes—their structure, their catalytic reactions, how their activities are regulated—is determined by BH4.

BH4 is also an essential cofactor for nitric oxide synthase (NOS), producing nitric oxide (NO). The latter actives guanylate cyclase, which in turn produces cyclic guanosine 3c,5c-monophosphate cyclic (cGMP).

Additionally, BH4 is a scavenger of oxygen-derived free radicals.

Although intestinal absorption of BH4 is adequate, humans do not obtain sufficient amounts of BH4 from dietary sources. The body must rely on de novo synthesis of BH4 to avoid deficiency.

A deficiency of BH4 leads to deficits in monoamine neurotransmitters (e.g., dopamine, epinephrine, norepinephrine, serotonin, and melatonin) and uncouples the catalysis of NOS. In the face of BH4 deficiency (or that of the substrate L-arginine, see below) the NOS-catalyzed oxidation of NADPH does not result in appropriate nitric oxide (NO) production, but instead enhances the generation of superoxide anion. The presence of the latter molecule in a milieu containing NO, permits rapid formation of peroxynitrite, the reactive species responsible for many independent toxic effects of induced NO.

However, peroxynitrite also oxidizes BH4 to quinonoid 5,6-dihydrobiopterin, which readily loses its side chain to form 7,8-dihydropterin. The latter is not a useful cofactor for eNOS. Thus, deficiencies of BH4 promote a cycle of self-destruction mediated by the formation of peroxynitrite. This mechanism is a significant contributor to the vascular endothelial dysfunction related to a variety of oxidative stresses.

To some extent L-arginine will reduce the generation of superoxide by NOS, but the inhibition of L-arginine on superoxide production is much weaker than that obtained with BH4, and a much higher concentration of L-arginine is required to attain a similar level of inhibition.

If adequate BH4 is available, eNOS produces physiologically appropriate amounts of NO from the eNOS substrate, L-arginine. But inadequate levels of BH4 lead, among other events, to endothelial dysfunction as a result of decreased production of this useful molecule. NO is involved via cyclic guanosine 3c,5c-monophosphate (cyclic GMP), either directly or indirectly, with many physiological signaling functions. These include among others: vasodilation, reduction of coagulation activity, and glucose transport into the cell via pathways parallel to, but distinct from those activated by insulin.

BH4 has been clinically investigated as therapy for phenylketonuria (PKC), Parkinson's disease, dystonia, depression, Rett syndrome, infantile autism, senile dementia, Alzheimer's disease and atherosclerosis. Although there have been provocative leads, except for PKC, the results have been discouraging. Perhaps this is because BH4 has been used as an isolated “silver bullet” (monotherapy)—it will be more effective as one segment of a therapeutic molecule or if it is administered in conjunction with LA, as described in this invention.

The de novo synthesis of BH4 produces three tetrahydropterins and one dyhydropterin, i.e., 6-lactyl-7′,8′-dihydropterin (sepiapterin). In some circumstances, these four molecules have been used with apparent success as stand-ins for BH4. All require sepiapterin reductase to catalyze their reduction to BH4. All are included as alternates to BH4 in this invention. The three tetrahydropterin molecules are:
6-1′,2′-dioxypropyl tetrahydropterin . . . 6-pyruvoyltetrahydropterin (Requires 6-pyruvolytetrahydropterin reductase; then sepiapterin reductase.)
6-1′-oxo-2′-hydroxypropyl tetrahydropterin . . . 6-lactoyltetrahydropterin (Also sometimes referred to as tetrahydrosepiapterin)
6-1′-hydroxy-2′-oxypropyl tetrahydropterin . . . 6-hydroxypropyl tetrahydropterin

B. Lipoic Acid

Lipoic acid—because of its lipophilicity and acidity, this is the trivial name of 1,2-dithiolane-3-pentanoic acid. The R configuration occurs naturally. The S configuration of lipoic acid, its racemic mixture, and the beta form are biologically less potent than the alpha form, particularly in the mitochondrion.

α-Lipoic acid (LA) is readily absorbed and distributed to all tissues where enzymes exist that can reduce lipoate to its more potent antioxidant form, dihydrolipoic acid (DHLA). Both DHLA and LA have metal-chelating capacity (providing antioxidant activity by chelating Fe2+ and Cu2+) and scavenge reactive oxygen species (ROS), whereas only DHLA is able to regenerate endogenous antioxidants and to repair oxidative damage.

In the latter case, it has been shown that dihydrolipoic acid (DHLA) reactivates oxidatively damaged alpha-1 antiprotease (alpha 1-AP). For the first time, it has been demonstrated that a drug (DHLA) is able to reverse oxidative damage of physiologically essential macromolecules. Previously, the only antioxidant properties that have been reported are those that prevent oxidative stress. Repair of oxidized alpha 1-AP is catalyzed by peptide methionine sulfoxide reductase (PMSR). DHLA acts as a reducing cofactor for PMSR. In addition to this ability to repair protease inhibitors, LA can prevent activation of the protease, caspase—the principle mediator of cellular apoptosis.

Thus, LA and reduced LA (DHLA) have been shown to have the potential for a curative as well as a preventative effect on human disorders.

LA also is 1) an essential dehydrogenase cofactor in energy metabolism, 2) a glutathione-sparing antioxidant and 3) a substrate for glutathione (GSH) synthesis, and 4) a potentiator of NO synthesis. The reduced form of LA, dihydrolipoic acid (DHLA) increases levels of GSH (reduced glutathione) in the cell, in part by reducing cystine to cysteine, which is utilized for GSH synthesis. LA normalizes GSH levels, but does not increase GSH beyond physiological levels. LA also prevents glutamate induced cellular damage, which is a factor of etiological importance in the neurodegenerative diseases addressed in this patent. In its “antioxidant role” LA scavenges hydroxyl radicals, hypochlorous acid, peroxynitrite, and singlet oxygen. In turn, GSH affects eNOS kinetics by recycling BH4 or preventing its autoxidation. And by scavenging peroxynitrite it further compliments BH4 by lessening the potential for cellular damage from toxic levels of induced NO.

LA coexists with GSH in the mitochondrion: this coexistence is very important, for example, in the modulation of dysfunctional apoptosis that is seen in the seemingly disparate neurodegenerative diseases addressed by this patent. Apoptosis is a prerequisite to any model of the developing nervous system. However, an increased rate of cell death in the adult nervous system underlies neurodegenerative disease and is one hallmark of multiple sclerosis, Alzheimer's disease, Parkinson disease and Huntington's disease.

LA potentiates endothelial NO synthesis (and thus, cyclic GMP bioactivity), in synergy with BH4, by mechanisms that appear to be, in part, independent of cellular GSH levels and the redox environment. Also, in concert with BH4, LA stimulates glucose uptake via cyclic GMP; the latter induces glucose transporter 4 (GLUT4) to move to the plasma membrane, facilitating glucose uptake into the cell. This action complements insulin, which induces GLUT4 activity via a separate tyrosine kinase mediated route. Remarkably, LA also directly activates tyrosine kinase, which increases glucose uptake in a manner similar to insulin. These properties are unique among all agents currently used to lower glycemia in animals and humans with diabetes.
Preferred dosage ranges (milligrams per day):

| | |
|---|---|
| Tetrahydrobiopterin bis-α-lipoate | 133 to 9056 |
| Tetrahydrobiopterin plus α-lipoate | 118 to 8050 |

### II. CLINICAL REVIEW

A. Diabetes Mellitus Type 1 (DB) and Type 2 (DB2), Impaired Glucose Tolerance Pre-Diabetes, Insulin Resistance (IR), Metabolic Syndrome X (MBS):

These related pathologies have reached epidemic proportions and affect about 30 to 45 million people (USA alone) and are growing rapidly. The clinical use of LABH4 as described in this patent is designed to be effective, as a standalone treatment for DB2/IR/MBS; as an adjunct to oral antidiabetics in DB2/IR/MBS; and as an adjunct to insulin in DB and DB2. Its use will reduce a variety of medical problems associated with this group of related pathologies:
1. Vascular endothelial dysfunction—Both IR and DB2 have a deficiency of BH4, as does MBS by definition; since MBS includes IR. In consequence they all (IR/MBS/DB/DB2) a have a significantly reduced activity of endothelial eNOS as a result of dysfunctional vascular endothelium—a notable feature of the serious vascular complications of IR/MBS/DB/DB2. In IR/MBS/DB/DB2, both serotonin (synthesized via TPH) and dopamine (synthesized via PAH & TH) are also reduced. These reductions most likely are secondarily associated with the BH4 deficiency.
Psychological depression and weight gain are common in IR/MBS/DB/DB2- and are associated with reduced serotonin levels. Perhaps it goes without saying, that in addition to lowering the quality of life, depression interferes with the patient's ability to manage their disease effectively.
2. CNS pathology—It is difficult to establish directly that cerebral dysfunction in DB2 is a result of decreased dopamine levels. It is known, however, that up to 80% of patients with Parkinson's disease are insulin resistant. This resistance to insulin is worsened by the now common use of L-dopa therapy. Thus, the potential for spiraling degeneration occurs: in the face of IR/MBS/DB/DB2, worsened by an existing BH4 deficiency, inadequate dopamine levels require more L-dopa supplementation, which in-turn worsens IR/MBS/DB/DB2, which reduces BH4 levels further, etc.
3. Glucose management—Glucose transport into skeletal muscle occurs via endocytic processes involving the glucose transport protein, GLUT4, which is translocated from an intracellular location into contact with and insertion into the plasma membrane. Importantly, this is mediated independently by either (or both) 1) insulin and 2) muscle contraction/exercise, suggesting that there are separate intracellular pools of GLUT4. In IR/MBS/DB/DB2 the GLUT4 pathway that is activated and modulated by insulin is defective. Because the alternate contraction/exercise GLUT4 pathway is dependent on available BH4, deficiencies of the latter negatively impact this backup, parallel glucose transport route. In the event of deficient BH4, therefore, even exercise is less effective in improving glucose transportation.
4. AMP-activated protein kinase (AMPK) is considered to be one link between exercise and glucose transport in muscle cells. It enhances GLUT4 glucose transport by activation of eNOS coupled to downstream signaling components, including cyclic GMP. BH4 is a rate limiting cofactor for eNOS.
AMPK is usefully stimulated by metformin. This demonstrates an interesting direct, potentially synergistic link and useful therapeutic relationship, between LABH4 and the widely used oral antidiabetic biguanide, metformin.
5. Linoic acid—LA has been shown to relocate GLUT4 to the plasma membrane, presumably at least in part energized via eNOS→cGMP. As mentioned above, LA augments BH4 and eNOS; primarily not because of either direct redox effects or indirect GHS effects, although both of these effects are likely involved to some degree.
In addition to its eNOS→cGMP→GLUT4 activity, LA has a GLUT4 stimulatory effect via tyrosine kinase and phosphatidylinositol 3-kinase activity; similar to the phosphorylation cascade caused by insulin. Thus, it appears that LA “works” both GLUT4 pools.
6. GTP cyclohydrolase inhibition—Pterins, especially reduced pterins like tetrahydrofolate, inhibit GTP cyclohydrolase—the initial and rate limiting enzyme in de novo BH4 synthesis (40). This underlines the importance of BH4 supplementation in patients with IR/MBS/DB/DB2 who are taking folic acid—a not uncommon clinical situation in this, usually, older age group.

B. Macrovascular Diseases, Including Nephropathy, Neuropathy and Retinopathy:

Most common microangiopathies occur frequently in diabetes. Impaired microcirculatory perfusion appears to be crucial to the pathogenesis of both neuropathy and retinopathy in diabetics. This in turn reflects a hyperglycemia-mediated perturbation of vascular endothelial function that results in: over-activation of protein kinase C, decreased BH4, reduced availability of NO, increased production of superoxide and endothelin-1(ET-1), impaired insulin function, diminished synthesis of prostacyclin/PGE1, and increased activation and endothelial adherence of leukocytes. This is ultimately a catastrophic group of clinical events.

Diabetic nephropathy, neuropathy and retinopathy represent major health problems, being responsible for substantial morbidity, increased mortality, and impaired quality of life. Near-normal glycemia is the primary approach to prevention of these conditions, but it is not achievable in a considerable number of patients.
1. Neuropathy—Nerve lipid peroxidation, the main cause of diabetic neuropathy, leads to deficits in neural blood flow, increases in neural oxidative stress, and pathological distal sensory nerve conduction. Neural oxidative stress appears to be due primarily to the dual processes of nerve ischemia and hyperglycemic autoxidation. These events can substantially be prevented by oral administration of LA. LA acts as a direct antioxidant but, importantly, it also preserves the critical thiol antioxidant GSH, which in turn favorably affects eNOS kinetics by recycling or preventing the autoxidation of BH4. BH4 has a protective effect against glucose neurotoxicity. The LA, GSH effect is very important, since GSH cannot effectively be administered orally as a supplement.
2. Nephropathy—Oxidative stress plays a central role in the pathogenesis and progression of the late microangiopathic complications in diabetic nephropathy. Albuminuria, an early sign of nephropathy, has been reduced in these patients when they are treated with LA.
Hyperglycemia causes depletion of LA in renal mesangial cells and compromises NO synthesis; a change that may play a role in the development of diabetic glomerulosclerosis. Worsening of the latter condition seems to parallel the reductions of BH4 that occur in diabetes. LA supplementation partially reverses this hyperglycemic effect. However, the addition of BH4 entirely restored the inducibility of NO synthesis.
3. Retinopathy—BH4 has been shown to reverse endothelial dysfunction in the ocular circulation in a diabetic rat model. The response to acetylcholine (an endothelium-dependent vasodilator mediated by stimulation of NO release) increases significantly in the presence of BH4.

C. Macrovascular Diseases, Including Heart Attack, Stroke, Peripheral Vascular Disease and Ischemia-Reperfusion Injury:
1. Reperfusion and LA—Ischemic-reperfusion injury in humans occurs in conditions such as stroke, heart attack, cardiac arrest, subarachnoid hemorrhage and head trauma. Tissue damage occurs during reperfusion primarily due to oxidative injury resulting from production of oxygen-derived free radicals (ROS). One of the major consequences of such damage is the depletion of the protective cellular antioxidant GSH, which leads to the oxidation transformation of protein thiols to disulfides. This lack of GSH is improved with LA treatment during ischemia-reperfusion. LA also reduces ATPase activity and increases ATP synthesis, further reversing the damaging outcomes of ischemia-reperfusion injury.
2. Heart—During episodes of myocardial hypoxia the administration of LA accelerates the recovery of aortic flow and stabilizes it during reoxygenation, protecting the myocardium from free radical-induced electrophysiological abnormalities, and decreasing the incidence of malignant arrhythmias.
3. CNS—There have been dramatic effects from LA in cerebral ischemia-reperfusion. In animal studies of cerebral ischemia: after LA pretreatment, there was a marked reduction in the mortality rate (from 78% to 26%) during 24 hours of reperfusion. This would seem to support the usefulness of LA in improving survival, and protecting the brain against reperfusion injury following cerebral ischemic episodes.
4. Reperfusion and BH4—A decreased level of BH4 aggravates endothelial dysfunction (see earlier discussion) and generally thereby indirectly contributes to cardiac and vascular dysfunction, reducing these tissues abilities to withstand both ischemia and reperfusion damage. There is also evidence that BH4 is effective for the direct treatment of ischemia-reperfusion injury. For example: A deficit in the endothelial production of NO is associated with the seriousness of sequelae that accompany reperfusion injury. However, after ischemia-reperfusion, the administration of BH4 restores the response of coronary arterioles to endothelium-dependent vasodilators. This suggests that ischemia-reperfusion alters the availability or production of BH4 itself, and the latter condition contributes to blunted endothelial nitroxidergic vasodilation.

The potential for therapeutic synergism between the molecular elements of this invention in the treatment of macrovascular disease is evident.

D. Hypertension, General Vasoconstriction, Nocturnal (Early AM) Vasoconstriction:

As previously stated, BH4 is a critical cofactor for eNOS. In its absence eNOS becomes “uncoupled,” producing ROS rather than NO. Thus, BH4 acts as a “redox switch”, decreasing superoxide release and enhancing appropriate levels of NO formation.

Insufficient BH4 and/or BH4 oxidation represents an important etiologic abnormality in systemic hypertension. The use of LABH4 will increase the availability of BH4.

LA has some antihypertensive effects associated with its antioxidative properties, which is probably governed by the normalization of superoxide anion production that occurs with LA treatment. This normalization in-turn spares BH4 from oxidation.

There is physiological balance between the properties of NO (vasodilation) and the vasoconstrictor ET-1 that mediates the autoregulation of blood flow. In a number of disorders there is a pathological shift in the balance toward ET-1 (vasoconstriction). An additional antihypertensive property of LA is that in concert with BH4 it potentiates appropriate endothelial NO synthesis, thereby favorably modulating NO/ET-1, tipping the balance toward physiological vasodilation.

The LABH4 of this invention has important inherent and systemic synergisms, some of these have been illustrated, and more will be pointed out. LABH4 will be effective in reducing vasoconstriction (including nocturnal vasoconstriction—of particular importance in cerebro- and cardiovascular events, and perhaps in the advancement of glaucomatous optic atrophy) in the treatment of hypertension and in preventing their associated vascular complications.

E. Obesity, Dyslipedemia:

A complex interaction between several neurotransmitters including dopamine, serotonin, neuropeptide Y, leptin, acetylcholine, melanin-concentrating hormone, ghrelin, nitric oxide, cytokines and insulin, determines and regulates food intake.

Leptin is a protein secreted by fat cells. It regulates body weight and thermogenesis in the brain. Blood-borne leptin reaches the brain via a saturable transport system located at the blood-brain barrier (BBB). Impaired BBB transport appears to underlie the resistance to the action of leptin that is seen in obesity. Leptin transport into the brain is enhanced 2-3-fold by epinephrine and by the catecholamine precursor and amino acid, tyrosine; each acts at the luminal side of the BBB.

Serotonin can produce weight loss. Indeed, the decreased serotonin found in DB2 is thought to contribute to obesity. Reuptake inhibitors of serotonin and noradrenaline, such as sibutramine, promote weight loss.

BH4 is the essential cofactor for the three aromatic amino acid hydroxylases phenylalanine (PAH), tyrosine (TH) and tryptophan (TPH), as well as for eNOS (as noted earlier). However, BH4 is often deficient in obesity, especially the obesity that is associated with DB2. This deficiency consequently results in reduced levels of tyrosine, epinephrine, dopamine, serotonin and NO (cGMP); all of these are established, modulating factors in weight regulation.

Tumor necrosis factor-alpha (TNF-α) is a cytokine involved in metabolic abnormalities and is overexpressed in the adipose tissue of obese rodents and humans. There is specific clinical evidence that TNF-α has a basic role in hypertriglyceridemia, glucose intolerance, and in the etiology of premature congestive heart failure—all of which are prevalent in diabetic patients. LA inhibits NF-kappaB activity, which in-turn limits TNF-α formation. An additional benefit is that LA reduces the expression of TNF-α-stimulated ICAM-1, and inhibits the expression of adhesion molecules, thus contributing to a reduction in endothelial cell/monocyte adherence and platelet adhesion.

F. Neurodegenerative Disorders, Including Parkinson's Disease, Mild Cognitive Impairment, Senile Dementia, Alzheimer's Disease, Hearing Loss and Chronic Glaucomas:

ROS are involved in a number of types of disorders of the brain and neural tissue. The metabolic antioxidant LA is a low molecular weight substance that is absorbed from the diet and crosses the blood-brain barrier. It affords both intracellular and extracellular oxidative protection. Both LA and dihydrolipoate (DHLA) are potent antioxidants, regenerating through redox cycling other antioxidants like vitamin C and vitamin E, and raising intracellular GSH levels. The most important thiol antioxidant, GSH, cannot be effectively orally administered—LA can. LA has been shown to have protective effects in cerebral ischemia-reperfusion, excitotoxic amino acid brain injury, mitochondrial dysfunction, diabetes and diabetic neuropathy, and other causes of acute or chronic damage to brain or neural tissue. LA has possible therapeutic roles in a variety of brain and neuronal tissue pathologies (some of these have already been discussed above). Studies indicate that LA has the potential to be effective in numerous neurodegenerative disorders (ND).

Of the many pathological factors involved in ND, decreased constitutive NO and increased ROS are common and related, and are favorably modified by this invention (as discussed above).

In many ND there is dysfunctional, premature apoptosis.

Mitochondrial decay and apoptosis associated with aging is in large part due to the oxidation of lipids and proteins, and of RNA and DNA. This increased oxidative damage to proteins and lipid membranes, particularly in mitochondria, causes a structural deformation of many enzymes, with a consequent decrease of enzyme activity as well as substrate binding affinity. Some of this mitochondrial decay can be reversed in aged animals by feeding the mitochondrial metabolite LA. This appears to restore mitochondrial function and delays mitochondrial decay and aging.

Both Alzheimer's disease (AD) and Parkinson's disease (PD) are associated with decreased BH4 and NO production. In these circumstances neurotoxic oxygen radicals may be produced. This is true in these and in other ND addressed by this invention. It will be recalled that if BH4 is insufficient, NOS becomes “uncoupled,” producing reactive oxygen species (ROS—notably superoxide) rather than NO. Additionally, BH4 makes dopaminergic neurons more resistant to oxidative stress caused by GSH depletion and, cooperatively, LA increases the GSH that is available to the neurons.

In neurodegenerative disorders, higher oral dosage levels of the LABH4 described by the patent (e.g., 40 to 60 mg/kg/day of BH4 bis-lipoate) may be required in part because of the status of the BBB. All available evidence predicts excellent safety at this dosage level.

Disorder-specific information follows, which underlines the usefulness of the invention:

### Alzheimer's Disease (AD)

BH4 metabolism is disturbed in AD and BH4 activity is decreased. Brains from subjects with AD retain their ability to synthesis neopterin and have normal dihydropteridine reductase activity, indicating a specific loss of ability to convert dihydroneopterin triphosphate to tetrahydrobiopterin. Because this is a critical de novo path for BH4 synthesis, supplemental BH4 is important.

Accumulations of peptides derived from beta-amyloid (Abeta) contribute to the etiology of AD by stimulating the formation of free radicals. Thus, an antioxidant such as alpha-lipoate, which is able to cross the BBB, is a logical choice for the treatment of AD. Investigations have shown that LA protects cortical neurons against cytotoxicity induced by Abeta (or by H₂O₂). In addition, LA induces an increase in protein kinase B/Akt in the neurons. Thus, part of the neuroprotective effect of LA is mediated through activation of the PKB/Akt signaling pathway.

### Parkinson's Disease (PD)

BH4, is the essential cofactor for phenylalanine hydroxylase and tyrosine hydroxylase, and as such is required for the synthesis of dopamine, the deficiency of which is the notable biochemical feature of PD.

BH4 is reduced in Parkinsonian striatum as is GTP cyclohydrolase I (GCH), the rate-limiting enzyme for BH4 biosynthesis. Thus, because of inadequate de novo BH4 synthesis in PD, supplemental BH4 is essential. Also, low BH4 synthesis raises the susceptibility of dopaminergic neurons to toxicities secondary to GSH depletion. Increasing BH4 levels does protect non-dopaminergic neurons. It would appear that reductions in BH4 levels may contribute to the pathogenesis of PD.

Oral BH4 has been unsuccessful in the treatment of PD. Probably it has been used in doses too small and/or in treatment durations too short to expect a favorable effect.

Depletion of GSH in the substantia nigra is one of the earliest changes observed in PD and could initiate dopaminergic neuronal degeneration. Data suggests that LA enters the brain and alters neuronal activity in areas of the basal ganglia intimately associated with the motor deficits exhibited by patients with PD. Whether this is due in part to LA's role in maintaining GSH levels is unclear.

### Hearing Loss

Noise-induced vasoconstriction with sludging of blood cells in the cochlea and the consequent accumulation of ischemic-reperfusion induced ROS is implicated in noise-induced hearing loss. Drugs that scavenge or block the formation of ROS, notably LA, protect the cochlea from damage resulting from the ischemic events caused by noise.

ROS cause extensive DNA, cellular, and tissue damage, which are all present with increasing frequency in presbyacusis. Mitochondrial DNA damage is the result of chronic exposure to ROS. LA can act in the mitochondria in reducing age-related hearing loss, reducing age-associated deterioration in auditory sensitivity, and in improving cochlear function. This effect appears to be related to LA's ability to protect and repair age-induced cochlear mtDNA damage, thereby upregulating mitochondrial function and improving energy-producing capabilities. In fact, even the artificially induced, and otherwise severe, ROS damage caused by aminoglycoside or cisplatin ototoxicity can be prevented or reduced by LA.

NO is involved in neurotransmission/neuromodulation in the cochlea. Under unfavorable ROS induced circumstances, NO (as peroxynitrite) becomes both a mediator of neurotoxicity and an initiator of apoptosis in the central nervous system and may play a role in noise induced ischemic processes in the cochlea. Again we should emphasize that when BH4 is insufficient, NOS becomes “uncoupled,” producing ROS (superoxide in this case) instead of NO, which in turn reacts with available NO to form the highly toxic peroxynitrite. A vicious circle occurs.

Hearing loss has long been associated with diabetes mellitus, being three to four times more prevalent in patients with DB2 than in subjects without diabetes. About half of DB2 patients have impaired hearing. This is another example of the interrelationships that exist between the seemingly disparate diseases addressed by this patent.

### Glaucoma

Chronic open angle glaucoma (COAG) is an optic neuropathy that progresses gradually toward blindness. Although elevated intraocular pressure (IOP) can damage the optic nerve mechanically, IOP fluctuation and blood pressure drops may lead to short-term ischemia, followed by reperfusion damage. LA has been shown to be effective in limiting neural ischemia-reperfusion damage. Likewise, it has been demonstrated that BH4 lessens ischemia-reperfusion injury, independent of its intrinsic radical scavenging action. Unfortunately ischemia-reperfusion alters the availability or production of BH4, which contributes to a blunting of an otherwise useful endothelial nitroxidergic vasodilation. The potential for synergism between LA and BH4 is again evident.

In COAG, glial cells secrete TNF-α leading to apoptotic death of retinal ganglion cells. LA reduces production of TNF-α by regulating NF-kappaB (see above).

The trabecular meshwork (TM) is the IOP outflow-controlling zone of the anterior segment of the eye. Oxidative stresses that disturb the TM may (probably) lead to elevation of the IOP in COAG. Inherent antioxidant defenses of ocular tissues are compromised even in the early as in the stages COAG. GSH is an important protective component of the cellular antioxidant system and it is reduced in glaucomatous eyes early in COAG. LA administration is associated with a rise of GSH levels in the red cells of patients with COAG. (Post LA treatment ocular levels of GSH were not determined in this study.)

Retinal ganglion cell death is the final common pathway of virtually all diseases of the optic nerve, including glaucomatous optic neuropathy. The retinal ganglion cells die after axonal injury (for a variety of reasons) often via the programmed cell death process of apoptosis. It has been found that dysfunctional apoptosis can be induced, in part, by ROS and an inadequacy of mitochondrial GSH.

LA administration is associated with a rise of GSH levels in patients with COAG and is associated with a lessening of ROS damage and apoptosis in the both the TM and the retinal ganglion cells.

The TM is highly enriched by the endothelial isoform of NOS. Abnormalities in NO or NO-containing cells are found in COAG. These abnormalities may be causally related to glaucoma. Such alterations—together with recent pharmacological studies showing that NO-mimicking nitro-vasodilators alter IOP—indicate that NO levels are relevant to the course and/or treatment of COAG. In fact, NO has emerged as an important endogenous inhibitor of apoptosis.

BH4 is one of the most potent naturally occurring reducing agents in addition to being an essential cofactor for the enzymatic activity of aromatic amino acid hydroxylases and eNOS. As mentioned above, suboptimal concentrations of BH4 reduce the formation of useful levels of NO and “uncouple” NOS leading to the formation of superoxide anions and hydrogen peroxide. Thus, NOS catalysis can result in either the formation of NO or of superoxide, depending on the presence or absence of BH4. Because NO reacts with the superoxide anion and hydrogen peroxide to form peroxynitrite, singlet oxygen, and the hydroxyl radical, any simultaneous release of NO and ROS in the presence of inadequate concentrations of BH4 is toxic. An increase in BH4 available to cells will reduce this dysfunctional NOS activity and protect local cells against consequent cell injury.

Endothelium-derived vasoactive substances are potent regulators of ophthalmic circulation, thus the important role of NO and ET-1 in the regulation of ophthalmic circulation. A disturbance in the modulating effects of these regulatory mechanisms has implications in the pathogenesis of glaucomatous optic atrophy.

BH4 has been shown specifically to enhance neuronal survival (of notable importance in glaucoma) in part by favorably altering Ca²⁺ cell-signaling mechanisms, and by BH4's effect on reducing reperfusion tissue damage. The favorable effect on reperfusion injury is mediated by enhancement of the NO/cyclicGMP pathway. Another complementary physiological interrelationship that is addressed by the invention.

Vascular insufficiency of the optic nerve may contribute to glaucomatous optic neuropathy, especially evident in glaucoma patients who have low or no elevation of IOP. In part at least this is due to chronic optic nerve head ischemia, which has been linked to the vasoconstrictor ET-1 and an impairment of peripheral NO mediated vasodilation.

ET-1 is also active in the anterior segment of the eye. The smooth muscle contraction produced by ET-1 strongly opposes the relaxation properties of NO and, as a result, trabecular contraction in the TM is stimulated, resistance to aqueous outflow is increased and IOP increases. At the same time that an increase of intraocular pressure occurs, there is vasoconstriction of the small vessels of the optic nerve; local hypoxia ensues and a course is set for optic nerve hypoxia, ischemia and atrophy. Aqueous levels of ET-1 are elevated in glaucomatous eyes; induced elevations of aqueous ET-1 levels have been shown to produce optic nerve collapse.

This balance between NO and ET-1 mediates the autoregulation of blood flow within the optic nerve as well as the peripheral circulation. Interestingly, the vascular reactivity of the peripheral circulation to ET-1 is much more pronounced in glaucoma patients than in non-glaucomatous subjects.

Metabolic Subsystem Disturbances

As mentioned earlier, several metabolic subsystem disturbances are globally applicable to each of the clinical targets of the invention. Each subsystem can be modified by the patent with a predictably favorable effect on of each of them, and with an equally favorable effect on the corresponding clinical target. In review—the common and modifiable subsystems that can be positively influenced by this invention are:

A. Mitochondrial Bioenergetics (& Apoptosis Control);

B. Free Radical Moderation (& Glutathione Maintenance);

C. Constitutive Nitric Oxide/Endothelin-1 Balance (Vascular Control);

D. Calcium Wave Signaling (& Neuronal Excitotoxicity).

A brief overview of metabolic subsystem disturbances, their complexity and more importantly their shared deficits, lead us to consider these interrelationships as they exist in the single representative pairing of Diabetes and Neurodegenerative disease. Similar examples of support for other combinations described by the patent also exist. Supporting concepts selected from the scientific literature would include:

Mitochondrial Bioenergetics (& Apoptosis Control)

Diabetes:
High glucose-induces endothelial cell apoptosis
Retinal neural cell apoptosis occurs early in diabetes
Beta-cell apoptosis is involved in the pathogenesis of human type 2 diabetes mellitus
Beta cell apoptosis rate is low in non-diabetic animals and increases 14-fold by 20 days after diabetes onset.

Neurodegenerative Diseases:
Neurodegenerative diseases have widely disparate etiologies but may share mitochondrial dysfunction as a final common pathway.
Mitochondria play an important role in mediating the initiation of apoptosis. A prolonged decrease in ATP levels caused by defects in oxidative phosphorylation underlies a number of neurodegenerative disorders.
Mitochondrial energy compromise could facilitate genetic abnormalities and enhance neuronal cell death. Such genetic abnormalities or mutations have been linked to various neurodegenerative diseases, that is, Huntington's disease, Alzheimer's disease, amyotrophic lateral sclerosis (ALS), etc.
There is substantial evidence that a mitochondrial deficiency of complex I activity might be involved in the pathogenesis of PD. This raises the possibility that agents that can modulate mitochondrial bioenergetics might exert neuroprotective effects

Free Radical Moderation (& Glutathione Maintenance)

Diabetes:
Oxidative stress is increased in diabetes, in various tissues, including nerve, kidney, and retina.
Pathogenesis of diabetic neuropathy . . . emerging data from human and animal studies suggest that glucose-derived oxidative stress has a central role, linking together many of the other currently invoked pathogenetic mechanisms
Although many risk factors can trigger the development of insulin-dependent diabetes (IDDM), it is likely that reactive oxygen species (ROS) play a central role in beta-cell death and disease progression.

Neurodegenerative Diseases:
There is significant evidence that the pathogenesis of several neurodegenerative diseases, including Parkinson's disease, Alzheimer's disease, Friedreich's ataxia and amyotrophic lateral sclerosis, may involve the generation of reactive oxygen species and mitochondrial dysfunction.
An important role for glutathione was proposed for the pathogenesis of Parkinson's disease, because a decrease in total glutathione concentrations in the substantia nigra has been observed in preclinical stages, at a time at which other biochemical changes are not yet detectable.
GSH depletion can enhance oxidative stress and may also increase the levels of excitotoxic molecules; both types of action can initiate cell death in distinct neuronal populations. Evidence for a role of oxidative stress and diminished GSH status is presented for amyotrophic lateral sclerosis, Parkinson's disease, and Alzheimer's disease.

Constitutive Nitric Oxide/Endothelin-1 Balance (Vascular Control)

Diabetes:
Hyperglycemia-induced upregulation of the ET-system in the heart is involved in the pathogenesis of cardiac problems in diabetes.
Upregulation of endothelin-1 appears to be a consequence of the nitric oxide-angiotensin II imbalance that contributes to end-organ injury, common to different diseases including diabetes mellitus.
High glucose-induced increased endothelial cell permeability may be induced through increased ET-1 expression and disorganization of F-actin assembly. ET-1 expression and increased permeability and may be modulated by nitric oxide.
These data indicate that in diabetes platelet Ca²⁺ signaling might be enhanced by excessive superoxide production and an attenuated negative direct or indirect feedback control by nitric oxide, due to its reduced production.

Neurodegenerative Diseases:
(NO→cGMP) . . . guanosine-3′,5′-cyclic monophosphate is a key mediator of neuroprotection
Inflammatory reaction is thought to be an important contributor to neuronal damage in neurodegenerative disorders such as Alzheimer's disease (AD), Parkinson's disease (PD), multiple sclerosis (MS), amyotrophic lateral sclerosis (ALS) and parkinsonism. Among the toxic agents released in brain tissues by activated cells is peroxynitrite, the product of the reaction between nitric oxide (NO) and superoxide. In the CNS it can be generated by microglial cells activated by pro-inflammatory cytokines or beta-amyloid peptide (beta-A) and by neurons in three different situations: hyperactivity of glutamate neurotransmission, mitochondrial dysfunction and depletion of L-arginine or tetrahydrobiopterin.

Calcium Wave Signaling (& Neuronal Excitotoxicity

Diabetes:
Abnormalities in Ca(2+)-handling proteins occur in diabetes mellitus.
In diabetes, platelet Ca2+signaling might be enhanced by excessive superoxide production and an attenuated negative direct or indirect feedback control by nitric oxide, due to its reduced production.
Advanced glycation end product (AGE) accumulation in a high glucose (HG) environment mediates some of the vascular complications of diabetes. AGE reduces Ca²⁺ release from intracellular stores and Ca²⁺ influx through plasma membrane channels

Neurodegenerative Diseases:
The etiology of neurodegenerative diseases (amyotrophic lateral sclerosis, Huntington's disease, Parkinson's disease, Alzheimer's disease) remains enigmatic; however, evidence for defects in energy metabolism, excitotoxicity, and for oxidative damage is increasingly compelling . . . . A complex interplay between these mechanisms exists. A defect in energy metabolism may lead to neuronal depolarization, activation of N-methyl-D-aspartate excitatory amino acid receptors, and increases in intracellular calcium, which are buffered by mitochondria. Mitochondria are the major intracellular source of free radicals, and increased mitochondrial calcium concentrations enhance free radical generation.
Glutamate-induced excitotoxicity has been implicated as an important mechanism underlying a variety of brain injuries and neurodegenerative diseases . . . glutamate-induced elevation of intracellular free calcium, [Ca(2+)](i).

Dosage forms and methods of use in accordance with this invention are for: a) The simultaneous administration of tetrahydrobiopterin (BH4), or a derivative or precursor thereof and lipoic acid (LA), or dihydrolipoic acid (DHLA), or a derivative or salt thereof or, b) the administration of a conjugate consisting of tetrahydrobiopterin bis-lipoate (TBL). As noted above, the invention also provides a novel method for the preparation of TBL.

In the preparation of the TBL conjugate it is necessary to protect the substituents on the pteridine ring prior to acylation of the hydroxyl groups on the propyl side chain. For this purpose we have used the t-butoxycarbonyl group.

After acylation with α-lipoic acid the protecting groups are removed with trifluoracetic acid (TFA).

The TFA treatment will leave the lipoic ester functions untouched. Only the bis-lipoate is prepared as it is not practical to prepare the mono-lipoate since the acylation conditions are not sufficiently selective and the separation of the resulting mixture of two mono acylates and the diacylate will be difficult and costly.

BH4+[(CH₃)₃COC═O]₂O→Tetra-t-butoxycarbonyl derivative+Lipoic Acid/DCC→Tetra-t-butoxy carbonyl bis-α-lipoate+Trifluoracetic Acid (TFA)→BH4 bis-α-lipoate

Thus, with the BH4 bis-D-α-lipoate, 1 g. of compound will deliver 0.39 g. of tetrahydrobiopterin (BH4) and 0.61 g. of D-α-lipoic acid. If racemic (+/−)-α-lipoic acid is employed in the synthesis the amount of biologically active lipoic acid will be reduced by one-half.

The invention defines dosage forms and methods of treatment for a) the simultaneous administration of tetrahydrobiopterin (BH4) or a derivative or precursor thereof and lipoic acid (LA), or dihydrolipoic acid (DHLA), or a derivative or salt thereof or, b) the administration of a conjugate consisting of tetrahydrobiopterin bis-lipoate (TBL) in clinical presentations of:
1. diabetes mellitus, including type 1 and type 2 diabetes, impaired glucose tolerance, pre-diabetes, insulin resistance, metabolic syndrome X and as an adjunct to oral antidiabetic agents and/or insulin;
2. microvascular diseases, including nephropathy, neuropathy and retinopathy;
3. macrovascular diseases, including heart attack, stroke, peripheral vascular disease and ischemia-reperfusion injury;
4. hypertension; vasoconstriction, including nocturnal (early AM) vasoconstriction;
5. obesity; dyslipedemia;
6. neurodegenerative disorders, including Parkinson's disease, mild cognitive impairment, senile dementia, Alzheimer's disease, hearing loss and chronic glaucomas.

### Preparation of Tetrahydrobiopterin Bis-Lipoate

As noted above, it is necessary to protect the substituents on the pteridine ring prior to acylation of the hydroxyl groups on the propyl side chain. For this purpose we have used the t-butoxycarbonyl group:

After acylation with α-lipoic acid the protecting groups are removed with trifluoracetic acid (TFA).

The TFA treatment will leave the lipoic ester functions untouched. Only the bis-lipoate is prepared as it is not practical to prepare a mono-lipoate because acylation conditions are not sufficiently selective and separation of the resulting mixture of the two mono acylates and the diacylate will be difficult and costly.

BH4+[(CH₃)₃COC═O]₂O→Tetra-t-butoxycarbonyl derivative+Lipoic Acid/DCC→Tetra-t-butoxycarbonyl bis-α-lipoate+Trifluoracetic Acid (TFA)→BH4 bis-α-lipoate

Thus, with the BH4 bis-D-α-lipoate, 1 g. of compound will deliver 0.39 g. of tetrahydrobiopterin (BH4) and 0.61 g. of D-α-lipoic acid. If racemic (+/−)-α-lipoic acid is employed in the synthesis the amount of biologically active lipoic acid will be reduced by one-half.

### EXAMPLE 1

### BH4+(O—C═O)2→Tetra-t-butoxycarbonyl derivative

### [6R (1R,2S)]-2-t-Butoxycarbonylamino-4-t-butoxycarbonyloxy-5,8-di-t-butoxycarbonyl-6-(1,2 dihydroxypropyl)-5,6,7,8-tetrahydropterdin (C29H47N6O11:MW 641.7)

A solution of 2.4 g. of 6-R-L-erythro-5,6,7,8-tetrahydrohydrobiopterin (Sigma-Aldrich), 9.6 g. of di-t-butyl dicarbonate, and 4.1 g. of triethylamine in 100 mL of dimethylformamide was kept at room temperature for 18 hr. and the warmed to 60 degrees for 2 hrs. After cooling the bulk of the solvent was evaporated under reduced pressure on the steam bath. Ethyl acetate (200 mL) was added and the resulting solution was 3× with 100 mL of cold 0.1N hydrochloric acid and several 100 mL portions of water to neutrality. The ethyl acetate solution was dried over sodium sulfate and evaporated to dryness under reduced pressure to yield 6.4 g. of the tetra-t-butoxycarbonyl derivative.

### EXAMPLE 2

### Tetra-t-butoxycarbonyl derivative+Lipoic Acid/DCC→Tetra-t-butoxycarbonyl bis-α-lipoate

### [6R (1R,2S)]-2-t-Butoxycarbonylamino-4-t-buoxycarbonyloxy-5,8-di-t-butoxycarbonyl-6-(1,2-bis-(+/−)α-lipoyloxypropyl)-5,6,7,8-tetrahydropterdin (C459H71N5O 13S4: MW 1018.1)

A solution of 6.4 g. of the tetra-t-butoxycarbonyl derivative from Example 1, 4.1 g. of (+/−)-α-lipoic acid (Aldrich Chemicals) and 0.3 g. of 4-pyrrolopyridine in 300 mL of methylene chloride was cooled with stirring in an ice bath. Dicyclohexylcarbodiimide (4.1 g) was added and the reaction mixture was stirred until esterification was complete as judged by tlc analysis. The N,N-dicyclohexylurea was filtered off and the filtrate was washed with 3×100 mL portions of 5% acetic acid solution and with several 100 mL portions of water to neutrality. The organic solution was dried over sodium sulfate and evaporated to dryness under reduced pressure to afford 9.7 g. of the bis-(+/−)-α-lipoyl derivative.

### EXAMPLE 3

### Tetra-t-butoxy carbonyl bis-α-lipoate+trifluoracetic acid →BH4 bis-α-lipoate

### [6R (1R,2S)]-2-Amino-6-(1,2-bis-(+/−) α-lipoyloxypropyl)-5,6,7,8-tetrahydro-4(1H)-pteridinone (6-R-L-erythro-5,6,7,8-tetrahydrohydrobiopterin 6-(1,2-bis(+/−)-α-lipoate) (C25H39N5O5S4: MW 617.6)

A solution of 5 g. of the bis-lipoate ester from Example 2 was dissolved in 100 mL of trifluoracetic acid and stirred at room temperature in a nitrogen atmosphere until cleavage of the t-butoxycarbonyl groups was complete. The trifluoracetic acid was evaporated under reduced pressure and the residue was dissolved in 200 mL of methylene chloride and the resulting solution was washed with 3×100 mL portions of 2% sodium bicarbonate solution and then with 3×100 mL of water until neutral. Evaporation of the solvent under reduced pressure gave 2.3 g. of product, which was purified by flash chromatography on silica gel using mixtures of ethyl acetate and methylene chloride as eluant. The fractions containing the bis-ester were pooled and the solvent removed under reduced pressure to afford 1.7 g. of the bis-(+/−)-α-lipoate.

### EXAMPLE 4

### Tetra-t-butoxycarbonyl derivative+Lipoic Acid/DCC 4 Tetra-t-butoxycarbonyl bis-α-lipoate

### [6R (1R,2S)]-2-t-Butoxycarbonylamino-4-t-buoxycarbonyloxy-5,8-di-t-butoxycarbonyl-6-(1,2-bis-(D)-α-lipoyloxypropyl)-5,6,7,8-tetrahydropterdin (C459H71N5O 13S4: MW 1018.1)

Repeating the procedure of Example 2 and replacing (+/−)-α-lipoic acid with D-α-lipoic acid furnished the D-α-lipoate derivative.

### EXAMPLE 5

### Tetra-t-butoxycarbonyl bis-α-lipoate+trifluoracetic acid→BH4 bis-α-lipoate

### [6R (1R,2S)]-2-Amino-6-(1,2-bis-(D) α-lipoyloxypropyl)-5,6,7,8-tetrahydro-4(1H)-pteridinone (6-R-L-erythro-5,6,7,8-tetrahydrohydro tetrahydrohydrobiopterin 6-(1,2-bis-(D)-α-lipoate) (C25H39N5O5S4: MW 617.6)

Repeating the procedure of Example 3 with the product of Example 5 as starting material gave the bis-D-α-lipoate.

### III. DEFINITIONS

All terms appearing in this specification and the appended claims are used in the same manner as commonly recognized among those skilled in the technology and terminology of pharmacology. These terms are therefore used in accordance with their conventional definitions, except as otherwise noted. Further clarifications of some of these terms as they apply specifically to this invention are offered below.

“Unit dosage form” refers to a composition intended for a single administration to a subject suffering from aging or a medical condition. Each unit dosage form typically comprises each of the active ingredients of this invention plus pharmaceutically acceptable excipients. Examples of unit dosage forms are individual tablets, individual capsules, bulk powders, liquid solutions, ointments, creams, eye drops, suppositories, emulsions or suspensions. Clinical alteration of a function or condition may require periodic administration of unit dosage forms, for example: one unit dosage form two or more times a day, one with each meal, one every four hours or other interval, or only one per day. The expression “oral unit dosage form” indicates a unit dosage form designed to be taken orally.

An “active agent” or “active ingredient” is a component of a dosage form that performs a biological function when administered or induces or affects (enhances or inhibits) physiological functions, conditions or processes in some manner. “Activity” is the ability to perform the function, or to induce or affect the process. Active agents and ingredients are distinguishable from excipients such as carriers, vehicles, diluents, lubricants, binders, buffers and other formulating aids, and encapsulating or otherwise protective components.

“Delivery vehicle” is a composition, which comprises one or more active agents, and is designed to release the active agent in a particular fashion, either by immediately dispersing the agents, or by releasing the agents in a slow sustained fashion. The term encompasses porous microspheres, microcapsules, cross-linked porous beads, and liposomes that contain one or more active ingredients sequestered within internal cavities or porous spaces. The term also includes osmotic delivery systems, coated tablets or capsules that include nonporous microspheres, microcapsules, and liposomes, and active agents dispersed within polymeric matrices. A dosage form can include one or more delivery vehicles.

“Controlled” or “sustained” or “time release” delivery are equivalent terms that describe the type of active agent delivery that occurs when the active agent is released from a delivery vehicle at an ascertainable and manipulatable rate over a period of time, which is generally on the order of minutes, hours or days, typically ranging from about thirty minutes to about 3 days, rather than being dispersed immediately upon entry into the digestive tract or upon contact with gastric fluid. A controlled release rate can vary as a function of a multiplicity of factors. Factors influencing the rate of delivery in controlled release include the particle size, composition, porosity, charge structure, and degree of hydration of the delivery vehicle and the active ingredient(s), the acidity of the environment (either internal or external to the delivery vehicle), and the solubility of the active agent in the physiological environment, i.e., the particular location along the digestive tract.

The phrase “substantially homogeneous,” when used to describe a formulation (or portion of a formulation) that contains a combination of components, means that the components, although each may be in particle or powder form, are fully mixed so that the individual components are not divided into discrete layers or form concentration gradients within the formulation.

### III. COMPOSITIONS, FORMULATIONS, AND DOSAGES

In general, the dosage forms of this invention contemplates the use powders, liquids, emulsions, immediate release tablets, sustained releases tablets, capsules, transmembrane delivery systems, electrophoretic delivery systems and other clinically effective forms of delivery.

The dosage forms of this invention can be formulated for administration at rates of one or more unit dosage forms per day, or one or more unit dosage forms at intervals longer than one day.

A. Single-Layer Tablets

In certain embodiments of the invention, the oral dosage form is a substantially homogeneous single layer tablet that releases all of its components into the stomach upon ingestion.

Oral unit dosage forms to be taken three to four times per day for immediate release tablets are preferred.

B. Sustained-Release Tablets

In certain other embodiments of the invention, the oral dosage form is a tablet in which the active agents are protected by an acid-resistant coating for release only in the intestine, and optionally in a sustained-release manner over a period of time.

The polymer matrix of the controlled (sustained) release tablet, having been given an enteric coating in the granulation process with EUDRAGIT, does not dissolve in the acid pH of the stomach, but remains intact until it passes to the upper part of the small intestine, where the enteric coating dissolves in the more alkaline environment of the intestine. The polymeric matrix then immediately begins to imbibe water from the intestinal fluid, forming a water-swollen gel. The agents incorporated into this layer are then available for intestinal absorption as they osmotically diffuse from the gel. The rate of diffusion the agent is reasonably constant for the useful life of the matrix (approximately four hours), by which time the incorporated agent is finally depleted and the matrix disintegrates. Such a single layer controlled release tablet, substantially homogenous in composition, is prepared as illustrated in the examples that follow.

The slower, more sustained release of the active agents can be achieved by placing the active agents in one or more delivery vehicles that inherently retard the release rate. Examples of such delivery vehicles are polymeric matrices that maintain their structural integrity for a period of time prior to dissolving, or that resist dissolving in the stomach but are readily made available in the post-gastric environment by the alkalinity of the intestine, or by the action of metabolites and enzymes that are present only in the intestine. The preparation and use of polymeric matrices designed for sustained drug release is well known. Examples are disclosed in U.S. Pat. No. 5,238,714 (Aug. 24, 1993) to Wallace et al.; Bechtel, W., Radiology 161: 601-604 (1986); and Tice et al., EPO 0302582, Feb. 8, 1989. Selection of the most appropriate polymeric matrix for a particular formulation can be governed by the intended use of the formulation. Preferred polymeric matrices are hydrophilic, water-swellable polymers such as hydroxymethylcellulose, hydroxypropylcellulose, hydroxyethylcellulose, hydroxymethylpropylcellulose, polyethylene oxide, and porous bioerodible particles prepared from alginate and chitosan that have been ionically crosslinked.

A delayed, post-gastric, prolonged release of the active ingredients in the small intestine (duodenum, ileum, jejunum) can also be achieved by encasing the active agents, or by encasing hydrophilic, water-swellable polymers containing the active agents, in an enteric (acid-resistant) film. One class of acid-resistant agents suitable for this purpose is that disclosed in Eury et al., U.S. Pat. No. 5,316,774 (“Blocked Polymeric Particles Having Internal Pore Networks for Delivering Active Substances to Selected Environments”). The formulations disclosed in this patent consist of porous particles whose pores contain an active ingredient and a polymer acting as a blocking agent that degrades and releases the active ingredient upon exposure to either low or high pH or to changes in ionic strength. The most effective enteric materials include polyacids having a pKₐ of from about 3 to 5. Examples of such materials are fatty acid mixtures, methacrylic acid polymers and copolymers, ethyl cellulose, and cellulose acetate phthalates. Specific examples are methacrylic acid copolymers sold under the name EUDRAGIT®, available from Rohm Tech, Inc., Maiden, Mass., USA; and the cellulose acetate phthalate latex AQUATERIC®, available from FMC Corporation, New York, N.Y., USA, and similar products available from Eastman-Kodak Co., Rochester, N.Y., USA.

Acid-resistant films of these types are particularly useful in confining the release of active agents to the post-gastric environment. Acid-resistant films can be applied as coatings over individual particles of the components of the formulation, with the coated particles then optionally compressed into tablets. An acid-resistant film can also be applied as a layer encasing an entire tablet or a portion of a tablet where each tablet is a single unit dosage form.

The dosage forms of the invention optionally include one or more suitable and pharmaceutically acceptable excipients, such as ethyl cellulose, cellulose acetate phthalates, mannitol, lactose, starch, magnesium stearate, sodium saccharin, talcum, glucose, sucrose, carbonate, and the like. These excipients serve a variety of functions, as indicated above, as carriers, vehicles, diluents, binders, and other formulating aids.

Oral unit dosage forms to be taken once or three times daily for controlled (sustained) release tablets are preferred.

The amounts of the primary components of the dosage forms of the pharmaceutical preparation of this invention can vary. Expressed in terms of milligrams per day some examples of components and preferred ranges are illustrated in the following Examples.

However, the following Examples are used for illustrative purposes and do not encompass the entirety of the formulations contemplated by the invention, i.e., they are not intended to limit the variety of formulation combinations contemplated by the invention.

### EXAMPLE 6

### Tetrahydrobiopterin bis-α-lipoate

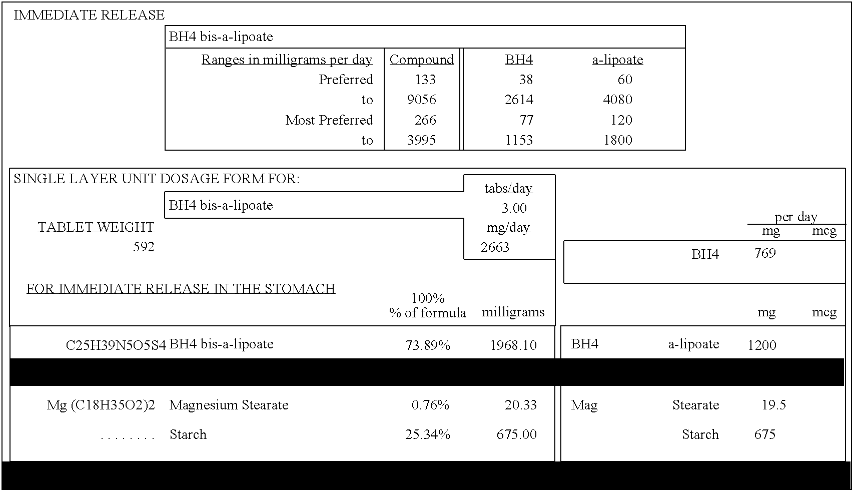

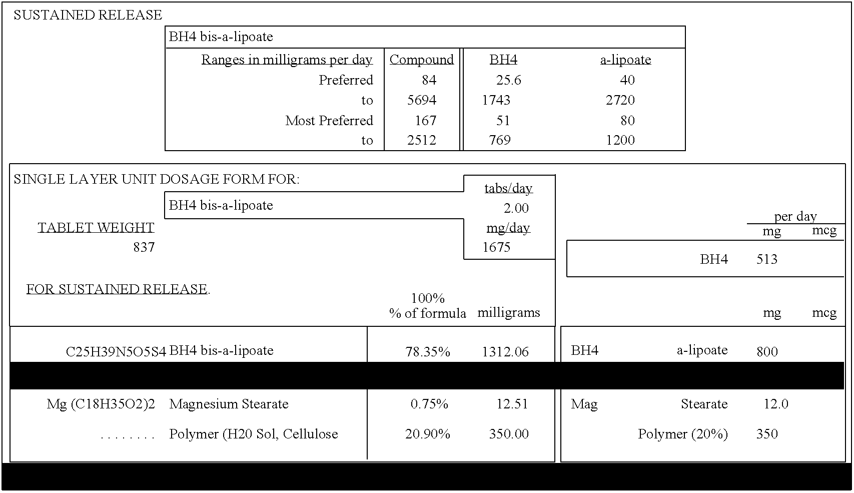

### EXAMPLE 7

### Tetrahydrobiopterin Plus α-lipoate

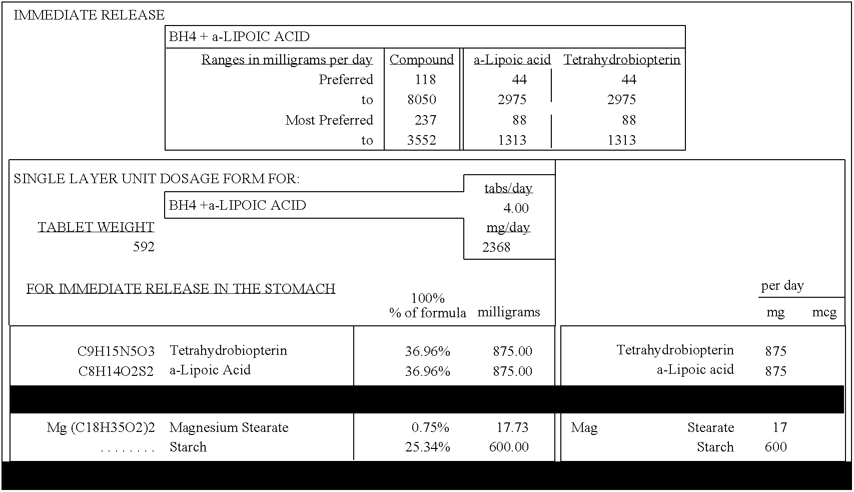

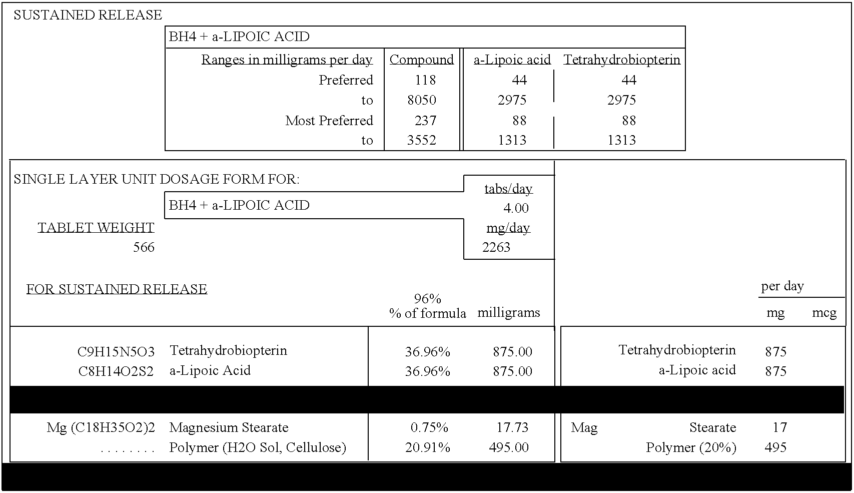

### REFERENCE LIST

The following references are hereby incorporated by reference for all legal purposes capable of being served thereby.
(1) Gorren A C, Mayer B. Tetrahydrobiopterin in nitric oxide synthesis: a novel biological role for pteridines. Curr Drug Metab 2002; 3(2): 133-157.
(2) Hoffmann H, Schmidt H H. Thiol dependence of nitric oxide synthase. Biochemistry 1995; 34(41):13443-52.
(3) Katusic Z S. Vascular endothelial dysfunction: does tetrahydrobiopterin play a role? Am J Physiol Heart Circ Physiol 2001; 281(3):H981-H986.
(4) Kay A D, Milstien S, Kaufman S, Creasey H, Haxby J V, Cutler N R et al. Cerebrospinal fluid biopterin is decreased in Alzheimer's disease. Arch Neurol 1986; 43(10):996-999.
(5) Kim Y M, Talanian R V, Billiar T R. Nitric oxide inhibits apoptosis by preventing increases in caspase-3-like activity via two distinct mechanisms. J Biol Chem 1997; 272(49):31138-31148.
(6) Kuiper M A, Visser J J, Bergmans P L, Scheltens P, Wolters E C. Decreased cerebrospinal fluid nitrate levels in Parkinson's disease, Alzheimer's disease and multiple system atrophy patients. J Neurol Sci 1994; 121(1):46-49.
(7) Landmesser U, Dikalov S, Price S R, McCann L, Fukai T, Holland S M et al. Oxidation of tetrahydrobiopterin leads to uncoupling of endothelial cell nitric oxide synthase in hypertension. J Clin Invest 2003; 111(8):1201-1209.
(8) Lee H A, Hughes D A. Alpha-lipoic acid modulates NF-kappaB activity in human monocytic cells by direct interaction with DNA. Exp Gerontol 2002; 37(2-3):401-410.
(9) Milstien S, Katusic Z. Oxidation of tetrahydrobiopterin by peroxynitrite: implications for vascular endothelial function. Biochem Biophys Res Commun 1999; 263(3):681-684.
(10) Nakamura K, Bindokas V P, Kowlessur D, Elas M, Milstien S, Marks J D et al. Tetrahydrobiopterin scavenges superoxide in dopaminergic neurons. J Biol Chem 2001; 276(37):34402-34407.
(11) Packer L, Tritschler H J, Wessel K. Neuroprotection by the metabolic antioxidant alpha-lipoic acid. Free Radic Biol Med 1997; 22(1-2):359-378.
(12) Packer L, Kraemer K, Rimbach G. Molecular aspects of lipoic acid in the prevention of diabetes complications. Nutrition 2001; 17(10):888-895.
(13) Seaton T A, Jenner P, Marsden C D. The isomers of thioctic acid alter C-deoxyglucose incorporation in rat basal ganglia. Biochem Pharmacol 1996; 51(7):983-986.
(14) Seidman M D, Khan M J, Bai U, Shirwany N, Quirk W S. Biologic activity of mitochondrial metabolites on aging and age-related hearing loss. Am J Otol 2000; 21(2):161-167.
(15) Stroes E, Kastelein J, Cosentino F, Erkelens W, Wever R, Koomans H et al. Tetrahydrobiopterin restores endothelial function in hypercholesterolemia. J Clin Invest 1997; 99(1):41-46.
(16) Tiefenbacher C P, Bleeke T, Vahl C, Amann K, Vogt A, Kubler W. Endothelial dysfunction of coronary resistance arteries is improved by tetrahydrobiopterin in atherosclerosis. Circulation 2000; 102(18):2172-2179.
(17) Vasquez-Vivar J, Martasek P, Whitsett J, Joseph J, Kalyanaraman B. The ratio between tetrahydrobiopterin and oxidized tetrahydrobiopterin analogues controls superoxide release from endothelial nitric oxide synthase: an EPR spin trapping study. Biochem J 2002; 362(Pt 3):733-739.
(18) Yu P K, Yu D Y, Cringle S J, Su E N. Tetrahydrobiopterin reverses the impairment of acetylcholine-induced vasodilation in diabetic ocular microvasculature. J Ocul Pharmacol Ther 2001; 17(2):123-129.
(19) Zhang L, Xing G Q, Barker J L, Chang Y, Maric D, Ma W et al. Alpha-lipoic acid protects rat cortical neurons against cell death induced by amyloid and hydrogen peroxide through the Akt signalling pathway. Neurosci Lett 2001; 312(3):125-128.
(20) Zhang W J, Frei B. Alpha-lipoic acid inhibits TNF-alpha-induced NF-kappaB activation and adhesion molecule expression in human aortic endothelial cells. FASEB J 2001; 15(13):2423-2432.

What is claimed is:

1. A unit dosage form for the management and clinical amelioration of a member selected from the group consisting of diabetes mellitus types 1 and 2, impaired glucose tolerance, pre-diabetes, insulin resistance, metabolic syndrome X, a microvascular disease, a macrovascular disease, hypertension, vasoconstriction, obesity, dyslipedemia, and a neurodegenerative disorder, said dosage form consisting of a bi-layer tablet comprising an immediate-release layer and a sustained-release layer, said tablet comprising a therapeutically effective amount of from about 11 mg to about 744 mg of tetrahydrobiopterin, and a therapeutically effective amount of from about 11 mg to about 744 mg of lipoic acid, with from about 40% to about 60% by weight of said tetrahydrobiopterin in said immediate-release layer and the balance in said sustained-release layer, and from about 40% to about 60% by weight of said lipoic acid in said immediate-release layer and the balance in said sustained-release layer.

2. A unit dosage form for the management and clinical amelioration of a member selected from the group consisting of diabetes mellitus types 1 and 2, impaired glucose tolerance, pre-diabetes, insulin resistance, metabolic syndrome X, a microvascular disease, a macrovascular disease, hypertension, vasoconstriction, obesity, dyslipedemia, and a neurodegenerative disorder, said dosage form consisting of a bi-layer tablet comprising an immediate-release layer and a sustained-release layer, said tablet comprising a therapeutically effective amount of from about 33 mg to about 2231 mg of tetrahydrobiopterin, with from about 40% to about 60% by weight of said tetrahydrobiopterin in said immediate-release layer and the balance in said sustained-release layer.

3. The unit dosage form of claim 1 wherein said tetrahydrobiopterin is a member selected from the group consisting of 6-lactyl-7′,8′-dihydropterin (sepiapterin), 6-1′,2′-dioxypropyl tetrahydropterin (6-pyruvoyltetrahydropterin), 6-1′-oxo-2′-hydroxypropyl tetrahydropterin (6-lactoyltetrahydropterin), and 6-1′-hydroxy-2′-oxypropyl tetrahydropterin (6-hydroxypropy)tetrahydropterin), (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin sepiapterin, 6-methyl-5,6,7,8-tetrahydropterin 6-hydroxymethyl-5,6,7,8-tetrahydropterin, 6-phenyl-5,6,7,8-tetrahydropterin, and precursors thereof.

4. The unit dosage form of claim 2 wherein said tetrahydrobiopterin is a member selected from the group consisting of 6-lactyl-7′,8′-dihydropterin (sepiapterin), 6-1′,2′-dioxypropyl tetrahydropterin (6-pyruvoyltetrahydropterin), 6-1′-oxo-2′-hydroxypropyl tetrahydropterin (6-lactoyltetrahydropterin), and 6-1′-hydroxy-2′-oxypropyl tetrahydropterin (6-hydroxypropy)tetrahydropterin), (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin sepiapterin, 6-methyl-5,6,7,8-tetrahydropterin 6-hydroxymethyl-5,6,7,8-tetrahydropterin, 6-phenyl-5,6,7,8-tetrahydropterin, and precursors thereof.

5. The unit dosage form of claim 1 wherein said lipoic acid is a member selected from the group consisting of alpha-lipoic acid, dihydrolipoic acid, and derivatives and salts thereof.
